(19) United States
(12) Patent Application Publication
(10) Pub. No.: US 2023/0184763 A1
(43) Pub. Date: Jun. 15, 2023

(54) METHOD OF DETECTING VIRUS HAVING HEMAGGLUTININ-ESTERASE ACTIVITY

(71) Applicant: Ji Hoon LEE, Gaithersburg, MD (US)

(72) Inventor: Ji Hoon LEE, Gaithersburg, MD (US)

(21) Appl. No.: 17/912,031

(22) PCT Filed: Mar. 16, 2021
(86) PCT No.: PCT/US2021/022602

§ 371 Date: Sep. 15, 2022

Related U.S. Application Data
(60) Provisional application No. 62/991,066, filed on Mar. 17, 2020.

(51) Int. Cl. G01N 33/569

(52) U.S. Cl. CPC G01N 33/56983; G01N 2333/165; G01N 2333/918

(57) ABSTRACT

A method of detecting a virus having a hemagglutinin-esterase activity in a sample is disclosed, the method includes: contacting the sample with a substrate for an enzyme of hemagglutinin-esterase (HE) of coronavirus or hemagglutinin-esterase fusion protein (HEF) of influenza C virus; and detecting activity of the enzyme, the detection of the activity indicates that the sample contains coronavirus or influenza C virus.

### CROSS-REFERENCE TO RELATED APPLICATION

The present application claims priority to and the benefit of U.S. Provisional Application No. 62/991,066, filed on Mar. 17, 2020, the disclosures of which is hereby incorporated herein by reference in its entirety for all purposes as if fully set forth herein.

### TECHNICAL FIELD

The present disclosure relates to a method for detecting virus having hemagglutinin-esterase activity.

### BACKGROUND ART

Coronaviruses are a group of related viruses that cause diseases in mammals and birds. In humans, coronaviruses cause respiratory tract infections that can be mild, and others that can be lethal, such as SARS, MERS, and COVID-19.

Coronavirus disease 2019 (COVID-19) is caused by severe acute respiratory syndrome coronavirus 2 (SARS-CoV-2). The World Health Organization (WHO) recognized COVID-19 as a pandemic on Mar. 11, 2020, and it is urgently necessary to have widespread and rapid testing capability. Testing is crucial as it allows the infected person to avoid infecting others and to quickly receive the care they need, and to slow down the spread of the disease.

There are several available methods for detecting coronavirus, including RT-PCR and coronavirus-specific antibodies (e.g., IgM/IgG) tests. However, these methods are labor and time intensive. Moreover, the testing method using the coronavirus-specific antibodies cannot distinguish an actively infected patient from a cured patient who still has antibodies after the infection is cleared.

An instant, inexpensive and accurate method of detecting coronaviruses, including COVID-19, from a human sample has yet to be developed, but is urgently needed. Such a test could save many lives from rapidly spreading and potentially fatal diseases.

### SUMMARY

The inventor recognized that hemagglutinin-esterase (HE) present on its surface is one of the unique characteristics of coronaviruses, including COVID-19 and Influenza C, when compared with other viruses having similar symptoms (e.g., rhinovirus for common cold, and Influenza A and B for flu), and completed the claimed invention, which provides a method of instant, inexpensive and accurate detection of a virus having an HE activity.

According to one aspect of the present invention, a method of detecting a virus having a hemagglutinin-esterase activity in a sample is provided, which includes: contacting the sample with a substrate for an enzyme of hemagglutinin-esterase (HE) of coronavirus or hemagglutinin-esterase fusion protein (HEF) of influenza C virus; and detecting activity of the enzyme, where detection of activity indicates that the sample contains coronavirus or influenza C virus.

The virus may be coronavirus, and the enzyme may be HE, and more specifically, the virus may be the coronavirus designated COVID-19. The enzyme may catalyze hydrolysis or deacylation of the substrate. In this method, the product of the hydrolysis or deacylation may be detected. The product formed from the deacylation or hydrolysis reaction between the substrate and the enzyme, may emit a detectable signal. The detectable signal may include fluorescence or chemiluminescence. The signal may be fluorescence, 1,1′-oxalyldiimidazole (ODI) chemiluminescence, or peroxyoxalate chemiluminescence (PO-CL). The substrate may be selected from among 4-Methylumbelliferyl acetate, Fluorescein diacetate, and Resorufin acetate. The sample may be a body fluid or a tissue sample from a human subject, and the sample is a nasopharyngeal or nasopharyngeal sputum sample. The sample and substrate may be incubated at room temperature with results showing almost immediately to 60 minutes, for example, depending on the amount of the sample per substrate. The incubation may be effected for at about 3 to 60 minutes. In the method, a plurality of readings of the activity of the enzyme may be taken as function of reaction time. A mixture of the sample and the substrate may have a pH of 4-10.

In accordance with another aspect of the present invention, a method of detecting coronavirus in a sample is provided, which includes: contacting the sample with an antibody of coronavirus; mixing the contacted sample with a substrate that reacts with an enzyme having hemagglutinin-esterase (HE) activity; and detecting activity of the enzyme, where the detection of activity indicates that the sample contains coronavirus. The antibody of coronavirus can be an antibody of SARS-CoV-2 or a specific variant thereof.

In accordance with another aspect of the present invention, a kit for detecting a virus having a hemagglutinin-esterase from a sample is provided, the kit comprises a container having a substrate that reacts with an enzyme having hemagglutinin-esterase (HE) activity, where the enzyme catalyzes hydrolysis or deacylation of the substrate, and a product of the hydrolysis or deacylation emits a detectable signal.

These and other aspects will be appreciated by one of ordinary skill in the art upon reading and understanding the following specification.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows SARS-CoV-2 (COVID-19) Structure (Cross-sectional model of a coronavirus shown, for example, in Wikipedia, https://en.wikipedia.org/wiki/Coronavirus).

FIG. 2 shows the reaction mechanism of ODI-CL and PO-CL using four different peroxyoxalate compounds. X: High-energy intermediate, Chemiluminescent probe under the ground state, and Chemiluminescent probe* under the excited state.

FIG. 3 shows hydrolysis reactions between a substrate and HE of coronavirus or HEF of influenza C. (1: 4-Methylumbelliferyl acetate, 2: 3-(2-Benzoxazolyl)umbelliferyl acetate, 3: Fluorescein diacetate, 4: Resorufin acetate, 5: 4-Methylumbelliferone, 6: 3-(2-Benzoxazolyl)umbelliferone, 7: Fluorescein, 8: Resorufin).

FIG. 4 shows fluorescence emission of 5: 4-Methylumbelliferone (4-MU), 6: 3-(2-Benzoxazolyl)umbelliferone, 7: Fluorescein, 8: Resorufin, formed from the hydrolysis reaction shown in FIG. 3.

FIG. 5 shows chemiluminescence of luminophore (5-8) emitted in peroxyoxalate (PO) or 1,1′-oxalyldiimidazole (ODI) chemiluminescence (CL) reaction.

FIG. 6 is a calibration curve capable of quantifying HE in a sample using the hydrolysis reaction of fluorescein diacetate and HE of coronavirus and chemiluminescence detection. (Relative CL intensity was measured with a LUMAT 9507 luminometer).

FIG. 7 shows hydrolysis reaction of HE and resorufin acetate under various concentrations of HE over time.

FIG. 8 is a photo showing naked-eye observation of the early diagnosis of coronavirus infection using the hydrolysis reaction of resorufin acetate and HE of coronavirus.

FIG. 9 is a calibration curve capable of rapidly sensing trace-levels of HE of coronavirus.

FIG. 10 shows a coronavirus captured by a spike antibody.

### DETAILED DESCRIPTION

The detection methods operated with fluorescence and chemiluminescence were developed to rapidly detect a specific virus in human samples based on the hydrolysis reaction between a substrate and hemagglutinin-esterase (HE) of coronavirus or hemagglutinin-esterase fusion protein (HEF) of influenza C. It should be understood that alternative signaling methods are contemplated beyond the specific examples provided herein

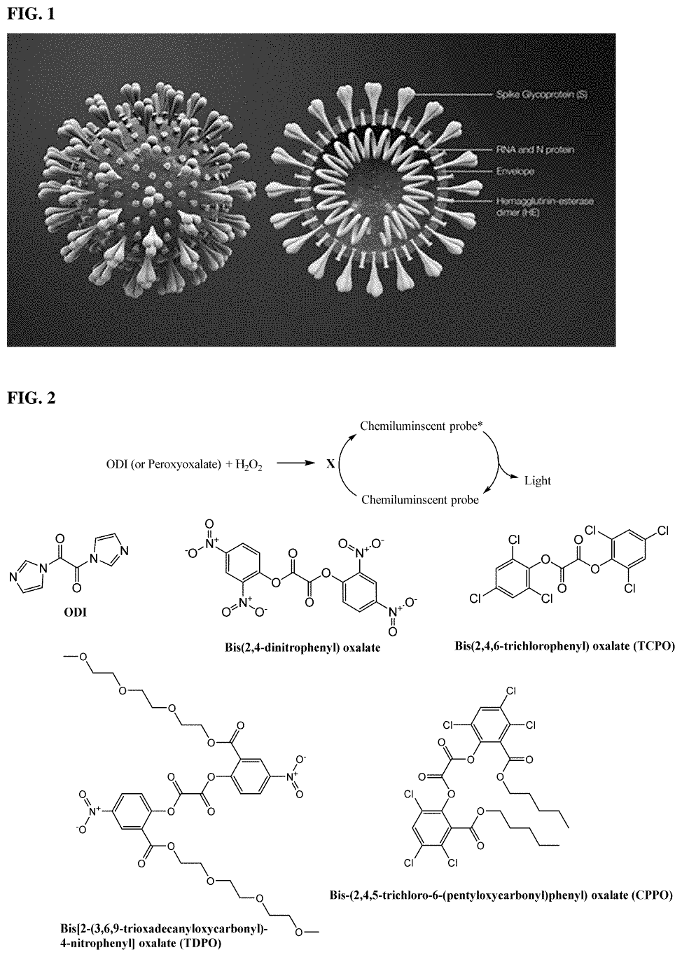

As shown in FIG. 1, coronavirus has many HEs with three different proteins (e.g., Spike glycoprotein, Nucelocapsid (N) protein, Envelope) and RNA. Also, two HEs (dimer) can be found on each of the spots of the coronavirus. HEF of influenza C is composed of three HEs (trimer).

The chemical and physical properties of esterase of HE and HEF are the same as those of acetyl (acetylxylan) esterase that catalyzes a chemical reaction, the deacetylation of xylans and xylo-oligosaccharides. Thus, because of the same chemical and physical properties, the inventor used acetyl (acetylxylan) esterase which can be easily obtained to show the results of the present invention. Moreover, the present inventor fully expects the same or similar results if HE or HEF are used.

Using the property of acetyl esterase, four different substrates 4-Methylumbelliferyl acetate, 3-(2-Benzoxazolyl)umbelliferyl acetate, Fluorescein diacetate, Resorufin acetate were synthesized and commercialized to monitor the activity of acetyl esterase in a sample. A product, formed from the deacetylation (or hydrolysis) reaction between acetyl esterase and a substrate, emits fluorescence, 1,1′-oxalyldiimidazole (ODI) chemiluminescence, and peroxyoxalate chemiluminescence (PO-CL).

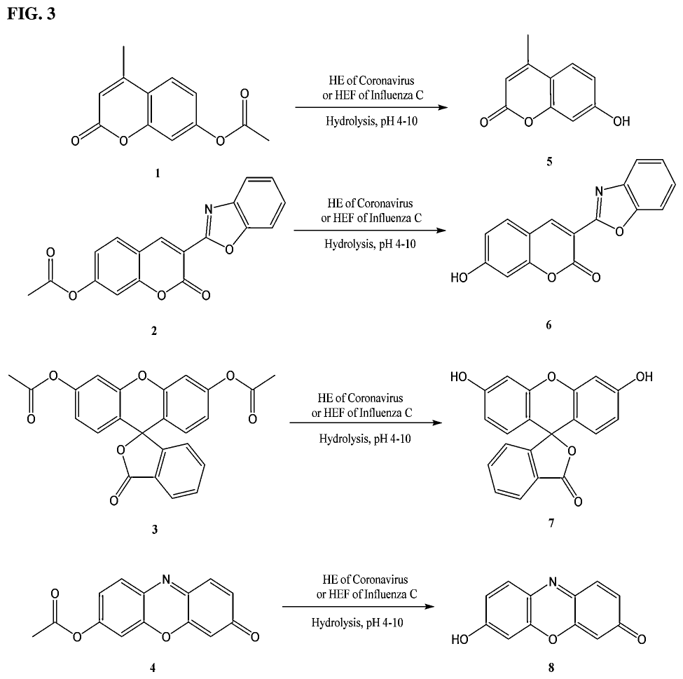

Based on the mechanism of deacetylation (or hydrolysis) reaction, novel analytical methods and kits have been designed and developed to early diagnose the infection of coronavirus or influenza C. Fluorescence, 1,1′-Oxalyldiimidazole chemiluminescence (ODI-CL) and peroxyoxalate chemiluminescence (PO-CL) detections were applied in this invention because the chemiluminescent probe, formed from the rapid hydrolysis reaction of a non-fluorescent & non-chemiluminescent substrate and HE of coronavirus (or HEF of influenza C), can emit light. High-energy intermediate(s), X, formed from the ODI- and PO-CL reactions act as the light source (Laser, LED, Xenon) to generate fluorescence as shown in FIG. 2.

Three different fluorescence compounds (5-8) were formed from the hydrolysis reaction of the substrate (1, 2, 3 or 4) and acetyl (acetylxylan) esterase (HE of coronavirus or HEF of influenza C existing in sputum, nasopharyngeal, nasopharyngeal, or saliva sample) as shown in FIG. 3. The yield of hydrolysis reaction is dependent on the pH (4-10), components of buffer solution, chemical & physical properties of substrate, and reaction (incubation) time of a substrate and HE (or HEF).

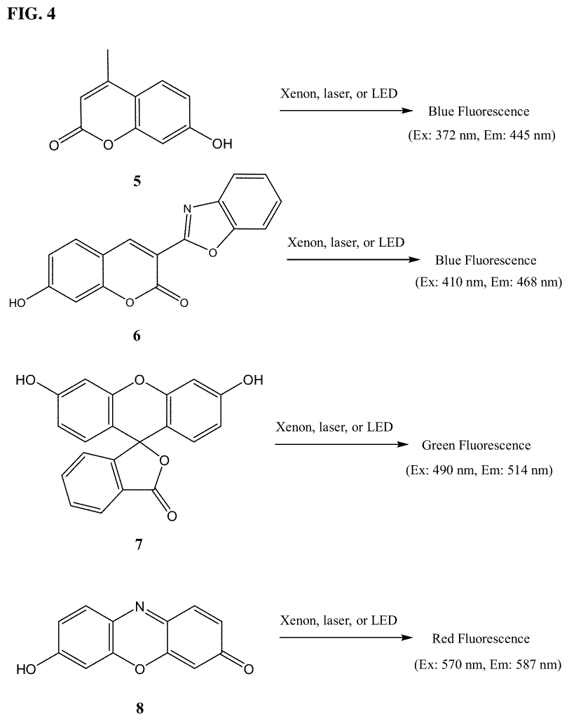

4-MU, 3-(2-Benzoxazolyl)umbelliferone, fluorescein, and resorufin formed from the hydrolysis reaction shown in FIG. 3 emit blue, green, and red fluorescence (see FIG. 4). With the increase of HE (or HEF) concentration in the hydrolysis reaction, relative fluorescence intensity of the product was proportionally increased. Using the simple method with fluorescence detection, it was confirmed that the novel analytical method can rapidly diagnose the disease of the patient infected with the coronavirus or influenza C. The diagnostic method with fluorescence detection was operated with a well-plate reader with fluorescence detection or a fluorometer operated with a cuvette cell or a borosilicate glass tube. A diagnostic kit was designed and developed based on the fluorescence detection method. The best incubation (reaction) time of a substrate and HE (or HEF) may be 3-30 min at room temperature to detect low concentration of coronavirus or influenza C in a sample. The sensitivity of the analytical method was dependent on the quantum efficiency of fluorescence compound formed from the hydrolysis reaction. In addition, the sensitivity of the analytical method with fluorescence detection was dependent on the concentration of the substrate. With the increase of substrate concentration, the sensitivity of the analytical method was enhanced. The range of the substrate used in this invention was 0.01˜0.5 mM. The components of the diagnostic kits are a substrate, reaction buffer, and a micro-well plate (e.g., 96, 384 micro-well plate) or a glass test tube.

As shown in FIG. 5, 1,1′-oxalalyldiimidazole (ODI) and several peroxyoxalate (PO) compounds have been used to invent the analytical method with chemiluminescence detection capable of early and rapidly sensing trace levels of coronavirus and influenza C. The peroxyoxalate compounds in this invention were bis(2,4-dinitrophenyl) oxalate (DNPO), bis(2,4,6-trichlorophenyl) oxalate (TCPO), bis[2-(3,6,9-trioxadecanyloxycarbonyl)-4-nitrophenyl] oxalate (TDPO) and bis-(2,4,5-trichloro-6-(pentyloxycarbonyl)phenyl) oxalate (CPPO). In order to obtain a rapid PO-CL reaction, sodium salicylate, pyridine, and an imidazole derivative such as imidazole, 2-methylimidazole, 4-methylimidazole, 2,4-dimethylimidazole, or 2-ethylimidazole, may be added as a base or nucleophilic catalyst.

The time necessary for completing the test may be dependent on the chemical and physical properties of a substrate shown in FIG. 3. Thus, the range of analytical time for quantifying coronavirus (or influenza C) in a sample may be from 1 to 30 min at room temperature. Additionally, the time necessary for producing chemiluminescent probes from the hydrolysis reaction shown in FIG. 3 was dependent on the property of buffer solution used to dissolve a substrate. Also, the analytical time will be dependent on the human sample collection method such as nasopharyngeal or oropharyngeal swab, nasopharyngeal or nasal aspirate and saliva collection. The reactivity of the hydrolysis reaction was dependent on the temperature. For example, the hydrolysis reaction at 37° C. was faster than that at room temperature.

The brightness of 4-MU, 3-(2-Benzoxazolyl)umbelliferone, fluorescein, and resorufin was dependent on the concentration of coronavirus or influenza C in a sample. With the increase of a virus concentration, the relative CL intensity was proportionally enhanced. The sensitivity of this method is dependent on the property of chemiluminescent probe formed from the hydrolysis of a substrate and HE (or HEF). The sensitivity of the method using resorufin formed from the reaction of resorufin diacetate and HE (or HEF) was better than those of 4-MU and fluorescein. The method can be operated with a luminometer or a well-plate reader with a CL detection.

As shown in FIG. 5, 4-MU, 5, emits blue or green light with the addition of PO or ODI with H₂O₂. The emission color of 4-MU was dependent on the pH used in the hydrolysis reaction shown in FIG. 3. The best buffer for the hydrolysis reaction of 4-methylumbelliferyl acetate and He (or HEF) was 1× PBS (pH 7.4). 4-MU formed in acidic condition (<pH 7) emits green light, whereas 4-MU formed in neutral and basic condition (pH 7 or higher) emits bright blue light. In addition, 4MU in basic condition was brighter than that in acidic condition. The time necessary for completing the analysis may be 30 min in PBS at room temperature. The concentration range of 4-methylumbelliferyl acetate may be 0.05˜0.5 mM for the hydrolysis reaction in this invention.

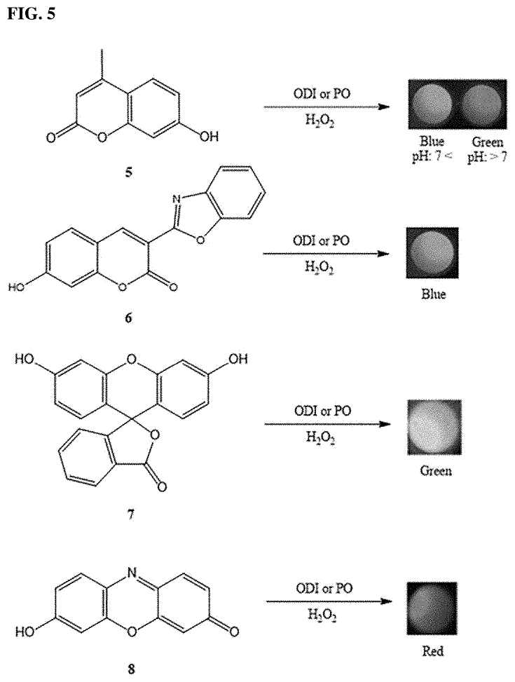

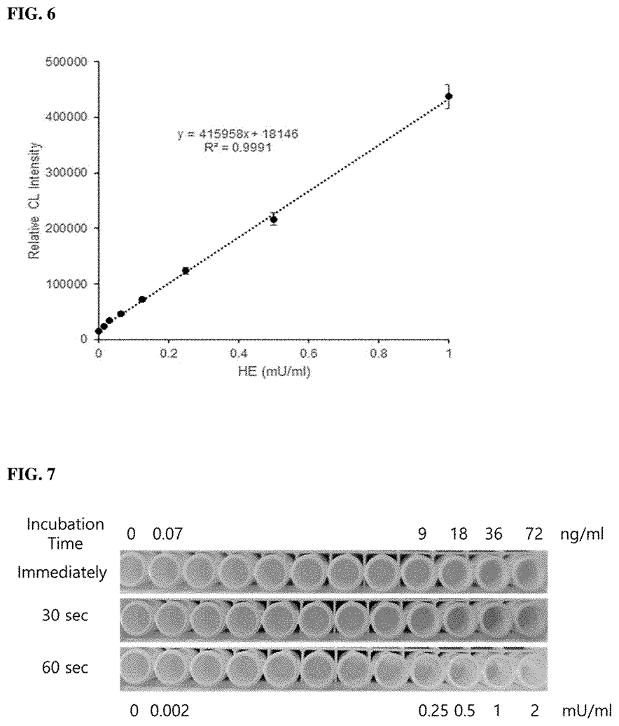

As shown in FIG. 5, 3-(2-Benzoxazolyl)umbelliferone, 6, emits blue light with the addition of PO or ODI with H₂O₂. The analytical method using fluorescein was more sensitive than that using 4-MU. Thus, the analytical time (5-10 min) using fluorescein diacetate was shorter than that using 4-methylumbelliferyl acetate. Also, 10 mM Tris-HCl buffer (pH 7-8.5) was used in this method. As shown in FIG. 6, the brightness of fluorescein was enhanced with the increase of HE in a sample. In this invention, the concentration range of fluorescein diacetate to produce fluorescein from the hydrolysis reaction was 0.01˜0.1 mM.

As shown in FIG. 5, fluorescein, 7, emits green light with the addition of PO or ODI with H₂O₂. The analytical method using fluorescein was more sensitive than that using 4-MU. Thus, the analytical time (10-20 min) using fluorescein diacetate was shorter than that using 4-methylumbelliferyl acetate. Also, 10 mM phosphate buffer (pH 8.5) was used in this method. As shown in FIG. 6, the brightness of fluorescein was enhanced with the increase of HE in a sample. In this invention, the concentration range of fluorescein diacetate to produce fluorescein from the hydrolysis reaction was 0.01˜0.2 mM.

As shown in FIG. 5, resorufin, 8, emits red light with the addition of PO or ODI with H₂O₂. The sensitivity of the analytical method using resorufin was better than those using 4-MU, 3-(2-Benzoxazolyl)umbelliferone, and fluorescein. Thus, the analytical time (1-5 min) using the resorufin acetate was faster than those using 4-methylumbelliferyl acetate and fluorescein diacetate. The best buffer solution in this method was 1× TBS (pH 7.5). The concentration range (0.001˜0.1 mM) of resorufin acetate for the hydrolysis reaction was much wider than those of other substrates such as 4-methylumbelliferyl acetate, 3-(2-Benzoxazolyl)umbelliferyl acetate, and fluorescein diacetate. The time necessary for quantifying HE (or HEF) in a sample collected using the nasoharyngeal swab or the oropharyngeal swab) can be very short. For example, the time necessary for quantifying acetyl (acetylxylan) esterase in a sample was as short as 3 min. In addition, the time necessary for quantifying samples collected using the nasopharyngeal swab can be less than 10 min. Additionally, the time necessary for quantifying saliva samples, which were diluted 20-fold with 1× TBS (pH 7.5), was as short as 5 min. The limit of detection (LOD=3S) determined with the analytical method was as low as 25 pg/ml. S is the standard deviation determined with background repeatedly measured 20 times. LOD of the analytical method with chemiluminescence detection was at least 10-fold lower than that with fluorescence detection. See FIG. 6. Also, the analytical method with chemiluminescence detection was at least 30 min faster than that with fluorescence detection because the former is much more sensitive than the latter.

With the increase of a virus concentration, the relative CL intensity was proportionally enhanced. The method can be operated with a luminometer or a well-plate reader with a CL detection.

As shown in FIG. 7, resorufin was formed from the rapid hydrolysis reaction between resorufin acetate and HE (or HEF). It was possible to observe resorufin (pink color), formed in the presence of relatively high concentration (>0.015 mU/ml or >0.56 ng/ml) of HE, with the naked eye within 60 sec. This result indicates that it is possible to rapidly observe with the naked eye whether a patient is infected from coronavirus (or influenza C). As evidence, FIG. 4 shows the color difference between 0.004 mU HE and the negative control not containing HE. FIG. 8 indicates that the early diagnosis of coronavirus can be possible with the naked eye.

As a tool for the naked-eye observation of coronavirus (or influenza C) infection, a lateral flow analysis (LFA) can be applied. The LFA applied in this invention is simpler than the conventional LFAs such as pregnancy, cancer, infection test kits because the former doesn't need any antibodies such as a capture or a detection antibody. For example, a sample (5˜100 μl) diluted in 1× TBS is loaded on the sample pad. The sample flows to the test line containing resorufin acetate. The test line changed color from white (or pale yellow) to strong pink with the rapid hydrolysis reaction when HE (or HEF) exists in the sample.

As shown in FIG. 9, chemiluminescence detection can quantify trace levels of HE in a sample. The result indicates that it is possible to early diagnose coronavirus infection using the analytical method with chemiluminescence detection within 3 min using the hydrolysis reaction of resorufin acetate and HE.

The components of the diagnostic kit designed based on the analytical method with chemiluminescence detection are a substrate (e.g., 4-methylumbelliferone (4-MU), 5, 3-(2-Benzoxazolyl)umbelliferone, 6, fluorescein, 7, resorufin, 8), reaction buffer, chemiluminescence reagents (e.g., ODI or PO, H₂O₂), and a 96 (or 384) micro-well plate or borosilicate glass tubes. Using the diagnostic kit prepared with resorufin acetate, for example, it is possible to conveniently and rapidly diagnose coronavirus or influenza C infection in the field, such as at public transport hubs such as airports, train stations, ports, and bus terminals.

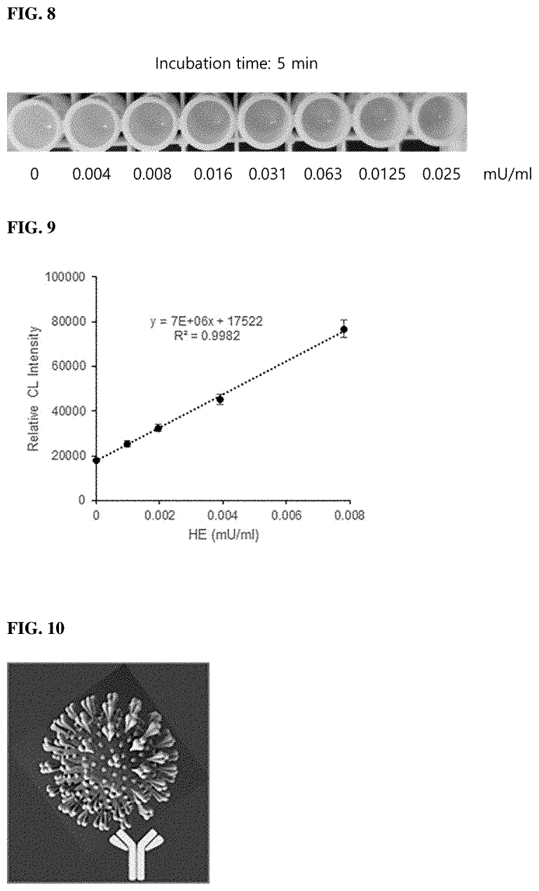

In order to confirm the infection of a specific coronavirus, an appropriate antibody, was applied in this invention as shown in FIG. 10. The antibodies, capable of capturing Spike glycoprotein or Nucelocapsid (N) protein of coronavirus, used in this method can bind with a protein of the coronavirus shown in FIG. 1. After capturing the coronavirus using the antibody, a substrate reacts with HE of coronavirus bound with the antibody for 10 min. Finally, using the CL measurement with the addition of chemiluminescence reagents, it is possible to confirm whether a patient is infected or not.

In conclusion, the analytical method with fluorescence or chemiluminescence detection can be applied to rapidly detect coronavirus or influenza C in a sample. This is because the accuracy, precision, and reliability of the method were good within a statistically acceptable error range.

It is to be understood that the above-described methods are merely illustrative embodiments of the principles of this disclosure, and that other compositions and methods for using them may be devised by one of ordinary skill in the art, without departing from the spirit and scope of the invention. It is also to be understood that the disclosure is directed to embodiments both comprising and consisting of the disclosed parts.

What is claimed is:

1. A method of detecting a virus having a hemagglutinin-esterase activity in a sample, comprising:
contacting the sample with a substrate for an enzyme of hemagglutinin-esterase (HE) of coronavirus or hemagglutinin-esterase fusion protein (HEF) of influenza C virus; and
detecting activity of the enzyme,
wherein detection of activity indicates that the sample contains coronavirus or influenza C virus.

2. The method of claim 1, wherein the virus is coronavirus, and the enzyme is HE.

3. The method of claim 2, wherein the virus is COVID-19.

4. The method of claim 1, wherein the enzyme catalyzes hydrolysis or deacylation of the substrate.

5. The method of claim 4, wherein a product of the hydrolysis or deacylation is detected.

6. The method of claim 5, wherein the product formed from the deacylation or hydrolysis reaction between the substrate and the enzyme, emits a detectable signal.

7. The method of claim 6, wherein the signal is color change of solution observed with naked eye, fluorescence or chemiluminescence.

8. The method of claim 7, wherein the signal is fluorescence, 1,1′-oxalyldiimidazole (ODI) chemiluminescence, or peroxyoxalate chemiluminescence (PO-CL).

9. The method of claim 1, wherein the substrate is selected from among 4-Methylumbelliferyl acetate, 3-(2-Benzoxazolyl)umbelliferyl acetate, Fluorescein diacetate, and Resorufin acetate.

10. The method of claim 1, wherein the sample is body fluid or tissue sample from a human subject.

11. The method of claim 10, wherein the sample is a nasopharyngeal, nasopharyngeal sputum, saliva sample.

12. The method of claim 1, wherein the sample and substrate are incubated at room temperature from less than 1 second to 60 minutes.

13. The method of claim 12, wherein the incubation is effected for at about 3 to 60 minutes for chemiluminescence detection.

14. The method of claim 12, wherein the incubation is effected for at about 20 to 60 minutes for fluorescence detection.

15. The method of claim 1, wherein a plurality of readings of the activity of the enzyme are taken as function of reaction time.

16. The method of claim 1, wherein a mixture of the sample and the substrate has a pH of 4-10.

17. A method of detecting coronavirus in a sample, comprising:
contacting the sample with an antibody of coronavirus,
mixing the contacted sample with a substrate that reacts with an enzyme having hemagglutinin-esterase (HE) activity; and
detecting activity of the enzyme,
wherein detection of activity indicates that the sample contains coronavirus.

18. The method of claim 17, wherein the antibody of coronavirus is an antibody of SARS-CoV-2 or a variant thereof.

19. A kit for detecting a virus having a hemagglutinin-esterase in a sample, comprising a container having a substrate that reacts with an enzyme having hemagglutinin-esterase (HE) activity,
wherein the enzyme catalyzes hydrolysis or deacylation of the substrate, and
wherein a product of the hydrolysis or deacylation emits a detectable signal.
